(19) United States
(12) Patent Application Publication
(10) Pub. No.: US 2015/0150930 A1
(43) Pub. Date: Jun. 4, 2015

(54) ARO-HONEY STARCH GEL FOR WOUNDS, BURNS AND SKIN CARE

(71) Applicants: Laïd BOUKRAA, Tiaret (DZ); Hasan A. ALZAHRANI, Jeddah (SA); Fatiha ABDELLAH, Tiaret (DZ); Balkees A. BAKHOTMAH, Jeddah (SA)

(72) Inventors: Laïd BOUKRAA, Tiaret (DZ); Hasan A. ALZAHRANI, Jeddah (SA); Fatiha ABDELLAH, Tiaret (DZ); Balkees A. BAKHOTMAH, Jeddah (SA)

(21) Appl. No.: 14/093,748

(22) Filed: Dec. 2, 2013

(51) Int. Cl. A61K 36/61; A61K 31/718; A61K 35/64

(52) U.S. Cl. CPC A61K 36/61; A61K 35/644; A61K 31/718

(57) ABSTRACT

Compositions comprising honey, starch hydrolysate, and clove EO are described. The compositions can also comprise one or more pharmaceutically acceptable diluents and/or excipients. Also described herein are methods for treating a wound comprising administering to the wound an effective amount of the present composition. The wound can be a burn, bed sore or a diabetic ulcer. The wound can also be infected.

### FIELD

The present application is generally directed to the field of medicine and pharmacology, particularly to compositions comprising honey, starch hydrolysate, and clove essential oil, and uses thereof for the treatment of wounds, burns, and general skin care.

### BACKGROUND

During the period following development of serious physical damage to the skin, the injured area is extremely unstable physiologically; following such injury or trauma, the normal physiological processes of the area in question may be severely compromised. The physician treating such damaged tissue must therefore be able to control and eventually reverse these undesirable effects, while at the same time stimulating the processes that are necessary to achieve healing of the area. Wound healing proceeds via unimpaired progression through overlapping phases: hemostasis, inflammation, proliferation, and remodeling; each phase is characterized by the infiltration into the wound site of specific cell types, all of which interact and communicate, by chemical signals, to optimize repair.

Because of the variety of different systems which may be adversely affected by such injury, it is difficult to find a single agent which will be effective in controlling the various sources of the problems. For example, one of the most severe difficulties encountered is the immediate colonization of the wound by a variety of different types of microbial species. Common invaders of a wound site are such known pathogens as Staphylococcus aureus, as well as a number of opportunistic pathogens, such as Escherichia coli or Pseudomonas aeruginosa. Various yeasts, particularly Candida albicans, may also be found at the wound. Although a number of antimicrobial agents for topical application are known, none has proven to be without some serious disadvantage. For example, silver sulfadiazine, the current antibacterial agent of choice is effective against gram-positive and gram-negative bacteria but many resistant strains have developed in the course of its use, particularly in the genus Pseudomonas. Similarly, the commonly used Betadine (povidone-iodine), although effective against both gram-positive and gram-negative bacteria, can be quite painful to the patient upon application, kills white cells in the wound, specifically polymorphonuclear cells, lymphocytes, monocytes, and macrophages, and may cause sensitization of an area already severely traumatized.

Therapeutic effects of honey have been found in wound treatment, by helping the rapid healing of wounds with less scarring and fighting infection. The healing effect of honey could be due to various physical and chemical properties. The high osmolarity and acidity of honey are among the physical characteristics that contribute to its antibacterial activity. Hydrogen peroxide, volatiles, organic acids, and flavonoids, are important chemical factors that provide the healing properties to honey. All these physical and chemical factors give honey unique properties as a wound dressing: it has a rapid clearance of infections, rapid debridement of wounds, rapid suppression of inflammation, minimization of scarring, and stimulation of angiogenesis as well as tissue granulation and epithelium growth. It has been reported to be particularly helpful in wound care of patients receiving chemotherapy, in whom the physiologic process of wound healing is impaired and prolonged. Many patents related to honey based formulations for wound management have been registered (see, e.g., European Patent Application No. 0 692 254 and U.S. Pat. No. 5,980,875).

Starch is one of the most abundant materials produced in nature, is easily recovered from plant organs holding it, is relatively low in cost, and is readily converted chemically, physically, and biologically into useful end-products. Starch is easily biodegradable, so formulations made from starch would, over time, be absorbed by the body. The therapeutic use of starch hydrolysate has been described in depth in U.S. Pat. Nos. 3,812,252 and 4,414,202. In brief, this material itself has been shown to be an exceptionally effective treatment for burns, ulcers, lesions, etc. The starch hydrolysate forms a film which ultimately adheres to underlying tissue and which is semipermeable to gas and fluid. It thus provides a covering which reduces plasma and fluid loss, while also preventing invasion by pathogenic microbes.

One of the characteristics that set honey apart from all other sweetening agents is the presence of enzymes. One of the most important honey enzymes is diastase, also called amylase. Diastase is added by the bee during the conversion of nectar to honey. The diastase content varies according to floral source, length of storage period, and exposure to high temperatures. Legislation has set a minimum level for diastase activity; generally it should not be less than 8 Diastase Number (DN) units, where 1 DN unit hydrolyses 1 mL of 1% starch using 1 g of honey for 1 hour at 37° C. The enzyme α-amylase splits the starch chain randomly, producing dextrin, and the enzyme β-amylase splits the reducing sugar maltose from the ends of the starch chain. The present inventors have found, in previous studies, that adding starch, which is the substrate of diastase, to honey has subsequently increased the antibacterial activity of honey, after incubation under specific conditions (see Boukraâ and Bouchougrane, Additive action of honey and starch against Candida albicans and Aspergillus niger, Revistalberoamericana de Micología 2007, 24(4):309-311; Boukraâ et al., Synergistic action of starch and honey against Aspergillus niger in correlation with Diastase Number, Mycoses 2008, 51:520-522; Boukraâ and Amara, Synergistic action of starch on the antibacterial activity of honey, Journal of Medicinal Food, 2008, 11(1):195-198; Boukraâ et al., Synergistic action of Starch and Honey against Pseudomonas aerugenosa in Correlation with Diastase Number, The Journal of Alternative and Complementary Medicine, 2008, 14(2):181-184; and Boukraâ et al., Additive Action of Starch and Honey against Candida albicans in correlation with Diastase Number, Brazilian Journal of Microbiology, 2008, 39:40-43).

The present inventors have also demonstrated, in vitro, that adding honey to essential oils (EOs) of Eugenia caryophyllata (clove) has synergistic activity against microbes (Boukraâ et al., Synergistic Effect of Monofloral Honeys and Essential Oils against Pseudomonas aeruginosa, British Microbiology Research Journal, 2013, 3(4):564-573; and Alzahrani et al., In vitro Synergistic Action of Honey and Essential Oils against two Species of Aspergillus: a Preliminary Study, British Journal of Pharmaceutical Research, In Press). Eugenol, eugenol acetate, and β-cariophyllene have been found in the essential oils of E. caryophyllata, and these compounds are described as having prominent antimicrobial property. Eugenol and clove oil have been used as local anesthetics in dentistry since the seventeenth century, specifically to reduce the pain associated with dental cavities.

### SUMMARY

Described herein are compositions comprising honey, starch hydrolysate, and clove EOs. In certain embodiments the composition is in the form of a gel (i.e. an aro-honey gel, or a gelatinous mixture comprising honey, starch hydrolysate, and clove EOs). The composition can also comprise one or more pharmaceutically acceptable diluents, carriers, vehicles and/or excipients. Preferably the starch hydrolysate is corn starch hydrolysate. The composition is generally produced by incubating a starch solution with honey prior to addition of the EOs and can have antiseptic, osmotic, and other beneficial characteristics.

Also described herein are methods for treating a wounds or other skin conditions in a subject in need thereof, comprising administering to the wound an effective amount of the present composition. In certain embodiments, the wound is an open or chronic wound, a burn, a bed sore or a diabetic ulcer. The wound can also be infected.

### DETAILED DESCRIPTION

Honey is a sweet food made by bees using nectar from flowers. The variety produced by honey bees (the genus Apis) is the one most commonly referred to and is the type of honey collected by beekeepers and used by humans. Honey bees form nectar into honey by a process of regurgitation and store it as a primary food source in wax honeycombs inside the beehive.

Honey gets its sweetness from the monosaccharides fructose and glucose and has approximately the same relative sweetness as that of granulated sugar. Honey has a long history of human consumption and is used in various foods and beverages as a sweetener and flavoring. It is also used in various medicinal traditions to treat numerous ailments. Most microorganisms do not grow in honey, at least in part because of its low water activity (aw) of about 0.6, acidic pH of about 3.2 to 4.5, and hydrogen peroxide content. In particular, honey is known to have antibacterial and antifungal activity against a broad spectrum of bacterial and fungal species.

Besides sugar, honey may also contain enzymes such as invertase, diastase (amylase), catalase, phosphatase, and glucose oxidase; organic acids such as malic acid, succinic acid, gluconic acid, acidic acid, and formic acid; inorganic acids such as phosphoric acid and hydrochloric acid; minerals such as Fe, Cu, P, S, K, Na, Mg, Ca, Si, Mn, Cl, and Zn; and, in small amounts, vitamins such as B1, B2, B6, pantothenic acid, nicotinic acid, H, folic acid, and vitamin C.

Amylases break down complex sugars, such as starch, into simple sugars. The α-amylases (alternative names: 1,4-α-D-glucan glucanohydrolase; glycogenase) break down long-chain carbohydrates by acting at random locations along the starch chain; the β-amylases (alternative names: 1,4-α-D-glucan maltohydrolase; glycogenase; saccharogen amylase) cleave off two glucose units (maltose) at a time working from the non-reducing end and catalyzing the hydrolysis of the second α-1,4 glycosidic bond.

Since the present composition may be directly applied to a wound, the honey that is used is preferably sterile. Sterile honey can be obtained by subjecting it to a sterilization treatment, for example sterilization by means of gamma rays. Experiments have shown that the honey retains its antibacterial activity after such radiation.

Starch is a carbohydrate consisting of a large number of glucose units joined by glycosidic bonds. Starch contains two types of polymeric glucose: amylose and amylopectin. Depending on the source, starch generally contains 20 to 25% amylose and 75 to 80% amylopectin by weight. Amylose consists of a linear chain of 200 to 1000 glucose residues linked by α-1,4-glycosidic bonds which cause the polymer to form a left-handed helix. Amylopectin contains up to two million glucose units and is a branched polymer of many amylose chains joined to each other at branch points by α-1,6-glycosidic linkages.

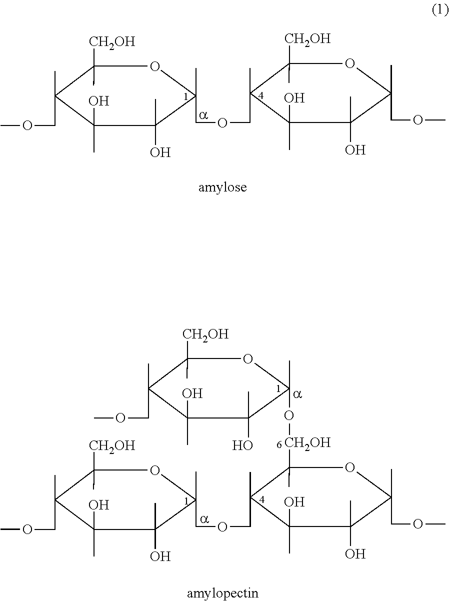

Amylose can be hydrolyzed to form maltose. The enzymatic hydrolysis of amylopectin yields a mixture of branched α-dextrins and maltose.

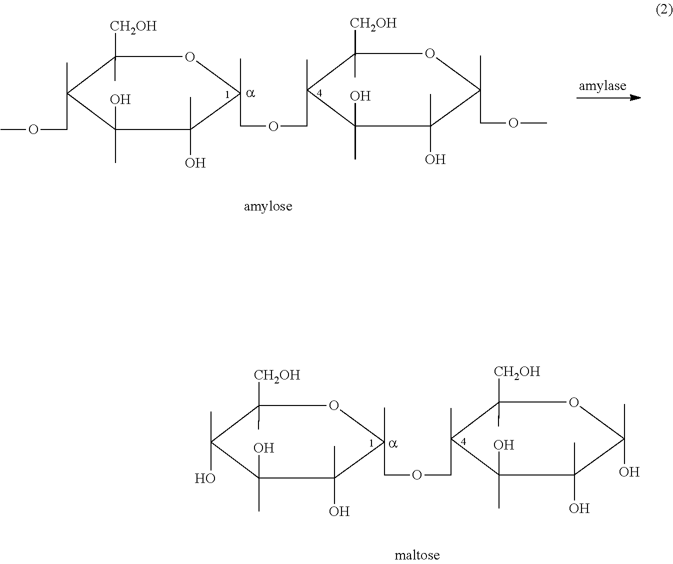

As described in U.S. Pat. Nos. 3,812,252 and 4,414,202, hydrolysis of starch produces a mixture of polymers of various molecular weights ranging from 200 glucose units or more down to maltose (2 glucose units) and D-glucose itself. Starch hydrolysate may be made by subjecting gelatinized starch to the hydrolytic action of an acid or/or an enzyme (Id).

The starch hydrolysate is not hydrogenated starch hydrolysate, which is produced by the partial hydrolysis of starch which creates dextrin (glucose and short glucose chains); the hydrolyzed starch (dextrin) then undergoes hydrogenation to convert the dextrin to sugar alcohols. A preferred starch hydrolysate is corn starch hydrolysate.

Eugenia caryophyllata (synonyms: Syzygium aromaticum, Caryophyllus aromaticus L., Eugenia aromatica L., Eugenia caryophyllus) is a tree (clove plant) in the family Myrtaceae. Eugenia caryophyllata essential oil (also known as oil of cloves or clove oil) is an essential oil from the clove plant, and is widely considered a natural analgaesic and antiseptic. There are four main types of clove essential oil: bud oil, derived from the flower-buds of E. caryophyllata and generally comprising 60-90% eugenol, eugenyl acetate, caryophyllene and other minor constituents; leaf oil, derived from the leaves of E. caryophyllata and generally comprising 82-88% eugenol with little or no eugenyl acetate, and minor constituents; stem oil, derived from the twigs of E. caryophyllata and generally comprising 90-95% eugenol, with other minor constituents; and mixtures of any two or more of the above. A preferred clove oil is bud oil.

In certain embodiments the composition is made by first hydrolyzing starch, for example by heating a starch solution to about 30-60° C. or 40-50° C. The starch solution is subjected to heating until the starch is dissolved, yielding an aqueous yellowish solution. The starch solution can comprise from 10-40%, preferably 20-30%, wt/v starch in water (e.g. distilled water). The resulting starch hydrolysate solution can be sterilized, for example by autoclaving. The starch hydrolysate solution can then be combined with honey at a ratio of about 1:1 v/v or about 20-30% (w/v) hydrolyzed starch to honey.

Honey contains amylase that, under suitable temperature and pH conditions (e.g. incubation at about 40-60° C., preferably 50° C.; at a pH of about 5-6, preferably pH 5.3-5.6; for about 2-6 hours, preferably 2-4 hours) will partially or completely degrade the starch hydrolysate into dextrin and maltose and therefore increase the antimicrobial activity of a honey and starch mixture. After the starch hydrolysate and honey mixture has been incubated, E. caryophylata EOs are added to the mixture at a ratio of about 1:10,000-1:20,000 (i.e. about 0.1-10 mls, preferably 0.5-1 ml, of EOs per 10 liters of honey/starch hydrolysate mixture).

A preferred ratio of starch hydrolysate:honey:clove EOs is about 1:1:1/10,000 (v/v/v).

The majority of conventional wound healing compositions suffer from one or another deficiency, whether complexity of application, insufficient ability to control infection, irritation caused to the patient, limited range of protective activity, or expense. To control infection the present composition possesses a dual synergistic activity against microbes. Furthermore, the composition forms a semipermeable continuous film backing on the wound that is optimized for oxygen and moisture vapor permeability and creates a barrier to liquids and infection. It has been reported that certain monosaccharides, when used either alone or in combination with many known wound treating compositions, may have the effect of providing added protection against microbial infection, enhancing the growth of granulation tissue, promoting the vascularization of the wound site, and/or stimulating the process of epidermal migration and wound closure (see, e.g., European Patent Application No. EP0221728A2). The main components of honey are monosaccharides, mainly glucose and fructose.

Also EOs of E. caryophylata will act synergistically with honey against microbes besides making the dressing non-painful. The whole mixture will form a film that prevent loss of fluids and plasma as well as prevent infection of the wound.

In certain embodiments the wound is covered with the present composition in an amount sufficient to allow formation of a film over the wound. The wound may then optionally be covered with a preferred non-adhesive dressing, which may be removed for the daily repeat of the treatment. This method of treatment is particularly applicable to mammalian skin wounds, and is most suitable for treatment of human wounds.

In certain embodiments the present composition draws fluid and debris from the wound bed and swells into a non-adherent gel, which helps reduce the risk of maceration. Additionally, the composition helps to ensure non-adherence to the wound bed so that the dressing can be removed without disturbing the healing tissues. E. caryophylata, besides boosting the antimicrobial effects of honey, may reduce pain in wounds during and after application of the dressing.

In a comparative study conducted on animal models with diabetic wounds (unpublished data), the present inventors have demonstrated that the present compositions have an improved overall effectiveness compared to other wound treatment preparations.

The present compositions may be indicated for the management of many wounds, including acute wounds, diabetic ulcers, leg ulcers, pressure ulcers (Stages I-IV), venous stasis ulcers, first and second degree burns, donor and graft sites, surgical wounds, skin lacerations, and dermatologic disorders.

Some advantages of the present compositions are as follows: 100% natural product; affordable and easily available; biodegradable; won't squeeze out of the wound as may honey alone may do; strong power of absorption; dry surface on the wound making a film (will sequestrate moisture inside); semipermeable, continuous film that is optimized for oxygen and moisture vapor permeability and as a barrier to liquids; easy to use; safe; will not stick to the wound bed; maintains integrity of healing tissues; will not dehydrate the wound bed; automatically donates moisture or absorbs exudate, depending on the condition of wound bed and fluid level in the dressing; helps relieve wound pain, inflammation, edema and bruising while improving comfort; could be inserted into cavities and sinuses; could serve as a base for other substances such as vitamins, minerals and amino-acids; and could also be used for many kinds of skin care (e.g. massage, make up, sunscreen).

Described herein are methods for treating wounds in a patient in need thereof by administering a therapeutically effective amount of the present composition. The compositions of the present invention can be administered topically, orally, parenterally, by inhalation (nasal or oral), vaginally, or rectally in dosage unit formulations containing conventional nontoxic pharmaceutically acceptable carriers, adjuvants, and vehicles, as desired. The term parenteral as used herein includes subcutaneous, intravenous, intramuscular, intrasternal injection, or infusion techniques.

The present composition can be formulated with only honey, starch hydrolysate, and clove EO. Alternatively, one or more pharmaceutically acceptable diluents, excipients or carriers can be added. For example, the composition can comprise a sterile aqueous solution to give a range of final concentrations depending on the intended use. Furthermore, the composition may comprise substances which assist in its application or storage stability, such as, e.g., stabilizers, preservatives, pharmaceutical adjuvants, water, buffer substances, thickening agents, emulsifiers, and the like. The techniques of preparing pharmaceutical compositions are generally well known in the art as exemplified by Remington's Pharmaceutical Sciences, 16th Ed. Mack Publishing Company, 1980. Moreover, for human administration, preparations should meet sterility, pyrogenicity, and general safety and purity standards, e.g. as required by the FDA Office of Biological Standards.

In certain embodiments, the compositions are topically administered. “Topical” administration here means local, external administration to a wound. The composition may be topically administered directly to all or to part of the wound or peripherally to the wound. Topical administration includes administration to the vulva and vagina.

For topical administration, for example, the compositions can be formulated as a gel, ointment, cream, balm, or lotion. Topical administration can also be accomplished with a liquid spray, an aerosol, or via iontophoresis, or through the use of liposomes, microbubbles and/or microcapsules. A composition suitable for topical administration can be formulated with only honey, starch hydrolysate, and clove EO. Alternatively, one or more pharmaceutically acceptable excipients and/or carriers can be added.

Gels, ointments and creams may be formulated, for example, with an aqueous or oily base with the addition of suitable thickening (e.g., wax, beeswax, PEG 4000, PEG 600, hard paraffin) and/or gelling agents (e.g., hydroxypropyl cellulose). Lotions may be formulated with an aqueous or oily base and can also generally contain one or more emulsifying agents (e.g., wool wax alcohol, fatty acid glycol esters), stabilizing agents (e.g., polyoxyethylene sorbitan monolaurate, carboxy methyl cellulose), dispersing agents (e.g., sodium oleate, propylene glycol), suspending agents (e.g., methyl cellulose, chitosan, accacia, carboxymethyl cellulose, tragacanth, pectin), thickening agents, and/or coloring agents (e.g., dyes, lackes). Other conventional pharmaceutical excipients for topical application include pluronic gels, polaxamer gels, hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (carbopols). Suitable carriers also include creams/ointments conventionally used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

The composition can be applied directly to the wound as a gel, ointment, liquid, cream, or the like as described above. Alternatively, the composition is administered in the form of a wound dressing. As used herein, the terms “wound dressing” and “dressing” refer broadly to any substrate when prepared for, and applied to, a wound for protection, absorbance, drainage, etc., and may include any one of the numerous types of substrates and/or backings that are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (non-woven composites of fibers from calcium alginate), and cellophane (cellulose with a plasticizer). For example, the composition can be applied to the surface of, or incorporated into, a solid contacting layer such as a dressing gauze or matrix. Suitable gauze dressings may include, for example, dry woven or non-woven sponges, swabs, bandages and wraps with varying degrees of absorbency. Exemplary fabric composition may include, for example, cotton, polyester or rayon.

In certain embodiments, gauzes and non-woven dressings may be available sterile or non-sterile in bulk and with or without an adhesive border. In certain embodiments the dressings also comprise one or more additional pharmaceutically active compound and/or carrier agent, including for example, saline, oil, zinc salts, petrolatum, xeroform and scarlet red.

While the compositions can be administered with honey, starch hydrolysate, and clove EO as the only active pharmaceutical agents in the methods described herein, they can also be used in combination with one or more compounds which are known to be therapeutically effective for wound treatment.

The present composition and/or the components of the composition may be sterilized by any suitable method, including conventional, well known sterilization techniques, such as autoclaving. Raw, unheated honey can be sterilized, for example, by ozonizing the honey using an ozone generator (see Vandeputte, U.S. Patent Application Publication No. 2010/0028408).

The dosage regimen for treating wounds is selected in accordance with a variety of factors, including the age, weight, sex, and medical condition of the patient, the severity of the wound, the route of administration, pharmacological considerations such as the activity, efficacy, pharmacokinetic and toxicology profiles of the particular composition used, whether a dressing or drug delivery system is used and whether the composition is administered as part of a drug combination.

The doses may be administered in single or divided applications. The doses may be administered once, or application may be repeated. Application may be repeated weekly or daily until wound healing is promoted, or a repeat application may be made in the event that wound healing slows or is stalled. Doses may be applied 1-7 days apart, or more. In the case of a chronic wound, repeat applications may be made, for example, one or more times per day, weekly, or bi-weekly, or monthly or in other frequency for example if and when wound healing slows or is stalled. For some indications more frequent dosing such as hourly application may be employed. The composition may be administered before, during, immediately following wounding, or later, for example when infection is diagnosed.

“Treatment” or “treating,” as used herein, refers to complete elimination as well as to any clinically or quantitatively measurable healing of the wound. Treatment may include, for example, promoting or accelerating wound healing, wound closure, and/or wound repair; reducing infection; reducing swelling and inflammation; and/or minimizing scar formation.

A “therapeutically effective amount” means the amount of a composition that, when administered to a subject for treating a wound, is sufficient to effect a desirable treatment for the wound. The “therapeutically effective amount” will vary depending on the particular composition, the wound and its type and severity, and the age, weight, etc., of the subject to be treated. A “therapeutically effective amount” need not result in a complete cure, but may provide partial relief of one or more symptoms or retard the progression of a condition such as infection.

“Patient” or “subject” refers to animals, and can include any mammal, such as human beings, rats, mice, cats, dogs, goats, sheep, horses, monkeys, apes, rabbits, cattle, etc. The mammalian subject can be in any stage of development including adults, children, infants, and neonates.

As used herein, the term “wound” includes an injury to any tissue, including, for example, acute, delayed or difficult to heal wounds, chronic wounds, and infected wounds. The term “wound” includes injuries to the skin and subcutaneous tissue initiated in different ways (e.g., pressure sores from extended bed rest and wounds induced by trauma) and with varying characteristics. The wounds amenable to treatment by the composition include injuries that can be located in any site, including external, interfacial, interstitial, extracorporeal, and/or intracorporeal. Also included are wounds that do not heal at expected rates. Examples of wounds may include both open and closed wounds. Wounds include, for example, cuts, gashes, lacerations, lesions, incisions, excisions, abrasions, surgical wounds, contusions, hematoma, crushing injuries, tissue rupture, Decubitus, Dermatitis, acute wounds, chronic wounds, battlefield wounds, necrotic wounds, necrotizing facitis, toxic epidermal nercolysis, pressure wounds, venous insufficiency ulcers, arterial ulcers, diabetic or neuropathic ulcers, pressure ulcers, mixed ulcers, burn wounds, Mucormycosis, Vasculitic wounds, Pyoderma, gangrenosum, and equivalents, and/or combinations thereof, known by persons skilled in the art.

Examples of wounds that do not heal at the expected rate can include ulcers such as diabetic ulcers, diabetic foot ulcers, vascultic ulcers, arterial ulcers, venous ulcers, venous stasis ulcers, burn ulcers, infectious ulcers, trauma-induced ulcers, pressure ulcers, decubitus ulcers, ulcerations associated with pyoderma gangrenosum, and mixed ulcers. Other wounds that do not heal at expected rates include dehiscent wounds. A delayed or difficult to heal wound may include, for example, a wound that is characterized at least in part by a prolonged inflammatory phase, a slow forming extracellular matrix, and/or a decreased rate of epithelialization or closure.

In certain embodiments the wound is infected with one or more of the following microorganisms: Staphylococcus aureus, Enterococcus faecalis, Pseudomonas aeruginosa, Escherichia coli, Candida albicans, Proteus mirabilis, Streptococcus pyogenes, Morganella morganii, Streptococcus pyogenes, Methicillin-resistant Staphylococcus aureus (MRSA), Vancomycin-resistant Enterococcus (VRE).

MRSA is a bacterium responsible for several difficult-to-treat infections in humans. It may also be called multidrug-resistant Staphylococcus aureus or oxacillin-resistant Staphylococcus aureus (ORSA). MRSA is, by definition, any strain of Staphylococcus aureus bacteria that has developed resistance to beta-lactam antibiotics which include the penicillins (methicillin, dicloxacillin, nafcillin, oxacillin, etc.) and the cephalosporins. MRSA is especially troublesome in hospitals or other health care settings, such as nursing homes and dialysis centers, where patients with open wounds, invasive devices and weakened immune systems are at greater risk of infection than the general public. VRE refers to several different species of antibiotic resistant Enterococcus bacteria that share similar characteristics and background. Similar to MRSA, people who are most susceptible for VRE infection are those in health care settings and/or with weakened immune systems.

As used herein, the singular forms “a”, “an” and “the” include plural referents unless the context clearly dictates otherwise.

The publications disclosed herein are provided solely for their disclosure prior to the filing date of the present application. Nothing herein should be construed as an admission that the present disclosure is not entitled to antedate such publication by virtue of prior disclosure. Further, the dates of publication provided may be different from the actual publication dates, which may need to be independently confirmed. All publications, patents, patent applications and other references cited herein are hereby incorporated by reference in their entirety.

While the disclosure has been described in detail with reference to certain embodiments thereof, it will be apparent to one skilled in the art that various changes can be made, and equivalents employed, without departing from the scope of the disclosure.

1. A composition comprising honey, starch hydrolysate, and clove essential oil (EO).

2. The composition of claim 1, wherein the honey:starch hydrolysate ratio is 1:1 based on volume.

3. The composition of claim 1, wherein the composition comprises about 1 ml of clove EO per about 10 liters of a honey and hydrolyzed starch mixture.

4. The composition of claim 1, wherein the honey:starch hydrolysate ratio is 1:1 based on volume and the composition comprises about 1 ml of clove EO per about 10 liters of the honey and hydrolyzed starch mixture.

5. The composition of claim 1, further comprising one or more pharmaceutically acceptable diluents and/or excipients.

6. The composition of claim 1, wherein the composition is in the form of a gel.

7. The composition of claim 1, wherein the composition is incorporated into a wound dressing.

8. A method for treating a wound in a subject in need thereof, comprising administering to the wound an effective amount of the composition of claim 1.

9. The method of claim 8, wherein the wound is selected from the group consisting of burns, bed sores and diabetic ulcers.

10. The method of claim 8, wherein the wound is infected.

11. The method of claim 10, wherein the wound is infected with one or more organisms selected from the group consisting of Staphylococcus aureus, Enterococcus faecalis, Pseudomonas aeruginosa, Escherichia coli, Candida albicans, Proteus mirabilis, Streptococcus pyogenes, Morganella morganii, Streptococcus pyogenes, Methicillin-resistant Staphylococcus aureus (MRSA), Vancomycin-resistant Enterococcus (VRE), and combinations thereof.
